(19) United States
(12) Patent Application Publication
(10) Pub. No.: US 2009/0248172 A1
(43) Pub. Date: Oct. 1, 2009

(54) POROUS MEMBRANE COMPRISING A BIOCOMPATIBLE BLOCK-COPOLYMER

(75) Inventor: Peter Neuenschwander, Baden (CH)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: EIDGENOSSISCHE TECHNISCHE HOCHSCHULE ZURICH, Zurich (CH)

(21) Appl. No.: 12/302,995

(22) PCT Filed: May 30, 2007
(86) PCT No.: PCT/EP2007/004773

§ 371 Date: Feb. 27, 2009

(30) Foreign Application Priority Data
Jun. 2, 2006 (EP) 06011457.6

(51) Int. Cl. A61L 27/44; C08L 53/00; B32B 27/28; B32B 1/08; A61F 2/02

(52) U.S. Cl. 623/23.75; 424/424; 521/137; 428/338; 428/36.5

(57) ABSTRACT

The invention relates to a membrane comprising a biocompatible block copolymer and has a porous structure with regularly distributed pores. A method for preparing said membranes is also provided.

### BACKGROUND OF THE INVENTION

The present invention relates to a membrane comprising a biocompatible block-copolymer obtainable by a polycondensation of at least two block units. A method for preparing said membrane is also provided.

Despite the vast number of polymers available nowadays there are only a few which are employed in the biomedical field. This holds especially true with respect to implants. The reasons for this situation are basically biocompatibility, mechanical properties, such as stiffness and elasticity, sterilizability, degradability of these polymers and a steadily growing number of administrative regulations in different countries which have to be met when using such polymers for medical purposes.

The process of formation of membranes is quite complex and difficult to control. In many cases, the resulting membranes are hardly or not permeable at all.

EP 0 196 486 discloses a biocompatible block copolymer which can be used as medical implant. This block copolymer has a crystalline and an amorphous component. The degradability of these block copolymers is, however, not fast enough for all applications.

EP 1 498 147 describes a biocompatible block copolymer with a controllable degradability. The block copolymer may also be employed in medical implants.

EP 1 452 189 discloses a shaped article comprising polyglycolic acid as a dimensionally stable carrier material. The surface of said carrier is coated with a biocompatible and degradable block copolymer. The shaped article may be used as an implant.

EP 1 452 190 describes an implant with a coating comprising a biocompatible block copolymer and on top of the block copolymer a polylysine layer.

US 2005/0155926 discloses a terpolymer made of tetrafluoroethylene, hexafluoropropylene and vinylidene fluoride. A classical method for producing a membrane comprising said terpolymer is also provided.

Membranes have been prepared by a variety of methods. A classical method is known as “non-solvent induced phase separation” (NIPS). In this method the polymer is dissolved in a solvent. A film of this solution is disposed on a carrier. The film is then contacted with a fluid which is a non-solvent for the polymer, but which is miscible with the solvent to induce phase inversion of the film.

Yet another method that has been used to prepare membranes is called “diffusion induced phase separation. The polymer solution is brought into contact with a coagulation bath. The solvent diffuses outwards into the coagulation bath while the non-solvent diffuses into the cast film. The exchange of solvent and non-solvent yields a solution which becomes thermodynamically unstable resulting in the separation of the components. A flat membrane is obtained.

The problem of the present invention is to provide membranes comprising a block copolymer which are biodegradable, have very good sterilizability and excellent mechanical properties, e.g. stiffness and elasticity and which have a uniform porous structure.

The problem is solved by a membrane according to the invention. Further preferred embodiments are also provided.

### BRIEF SUMMARY OF THE INVENTION

The present invention relates to a membrane comprising a biocompatible block-copolymer obtainable by a polycondensation of at least two block units. A method for preparing said membrane is also provided.

The term medicine or medical as used herein means both human and veterinary medicine.

Membrane as used herein means a typically planar, porous structure. However, the membrane can also have different shapes, e.g. a tubular shape or be a hollow fibre.

The term “pore” as used herein means a minute space in a material. The minute space has a highly complex and irregular form.

Mesh as used herein means a typically planar network comprising fibers that are interconnected in regular or irregular manner.

### BRIEF DESCRIPTION OF THE SEVERAL VIEWS OF THE DRAWINGS

In the following the figures are briefly described:

FIG. 1A illustrates the step of sucking the polymer solution into a tube preparing a membrane having a tubular form using the “negative method”.

FIG. 1B shows a further step of the “negative method” where the tube with the remaining polymer solution is horizontally mounted and rotated in order to achieve an even layer of the polymer.

FIG. 2 illustrates the porous structure of a membrane prepared from 16% polymer solution (solvent dioxan).

FIG. 3 shows the porous structure of a membrane prepared from 14% polymer solution (solvent dioxan).

FIG. 4 shows illustrates the porosity of a membrane prepared from 14% polymer solution (solvent dioxan)

FIG. 5 shows a membrane prepared from 14% polymer solution (solvent dioxan).

FIG. 6 shows a cross section of a membrane.

FIG. 7 shows a cross section of a membrane at a higher magnification.

FIG. 8 illustrates a sheet-like structure comprising a membrane and a mesh.

FIG. 9 again shows a sheet-like structure.

FIG. 10 illustrates the combination of a membrane and a mesh forming a sheet-like structure.

FIG. 11 shows a sheet-like structure comprising a membrane and a mesh at a higher magnification.

FIG. 12 shows a sheet-like structure at a high magnification.

FIG. 13 shows a welding seam of two membranes forming a bag-like structure.

FIG. 14 shows a section of two membranes which have been welded.

FIG. 15 shows a welding seam of a membrane capsule.

FIG. 16 shows fibroblast 3T3 cells after 72 h in the eluate (a) control (b) membrane.

FIG. 17 shows a fluorescein diacetate staining of live cells on membrane, 72 h.

FIG. 18 shows a fluorescein diacetate staining of live cells on membrane, 12 d.

### DETAILED DESCRIPTION OF THE INVENTION

The membranes of the present invention comprise a block copolymer which is highly biocompatible and also biodegradable. The degradability can be precisely controlled by minor changes in the chemical composition of the membrane. The base material of the membrane is a pure polymer. No further additives such as stabilizers, antioxidants or plastifiers, which could adversely affect the excellent biocompatibility, are needed. Further advantageous properties are their elasticity while still maintaining excellent mechanical stiffness. The mechanical properties strongly depend on the crystalline and the non-crystalline compound.

Membranes according to the present invention have porous structure. Pores are present throughout the entire membrane and they are regularly distributed in the membrane resulting in a regularly structured membrane. The membranes are considered as symmetric, since the interfaces on both sides show the same structures, with only minimal differences compared with the structure of the inside. In addition, the average pore size varies only within a limited range. The porosity and the pore size of the membrane of the present invention can be varied according to needs of the intended use making the membranes a very versatile tool. Porosity can on one hand be controlled by the concentration of the block copolymer solution and on the other hand by the choice of the solvent. An increasing concentration leads to a decrease of the pore size.

It is possible that liquids and (macro-)molecules diffuse into or even pass through the membrane of the present invention. This permeability, obeying Fick's equation, is a great advantage, if the membrane is used in biological systems. There it is very important that artificial membranes allow the exchange of gases, e.g. oxygen, liquids and compounds, e.g. nutrients for and waste of cells. If such exchange is hampered or not possible at all, cells which are in close contact with such membranes may die.

In a preferred embodiment membranes according to present invention are permeable or semipermeable.

The permeability depends on the size of the pores.

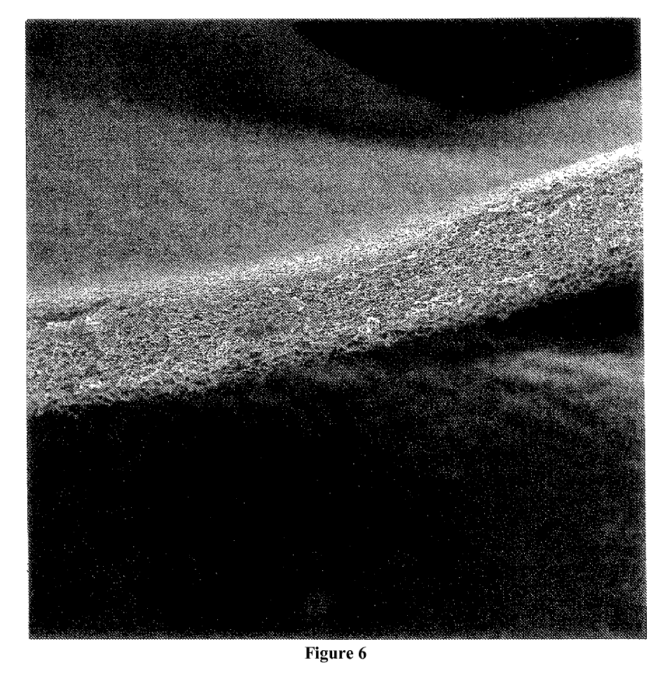

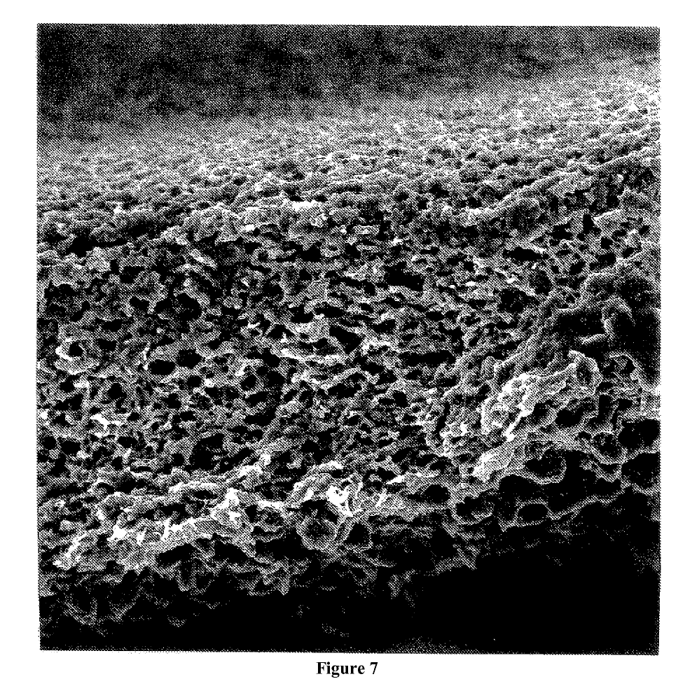

Membranes of the present invention have pores in a size range of 0.2 to 20.0 μm, preferably in the size range of 0.2 to 10.0 μm. The permeability for such membranes (e.g. FIG. 6) defined as

$$P = \frac{( amount of permeant ) ( membrane thickness )}{( area ) ( time ) ( driving force gradient across membrane )}$$

The permeability of membranes according to the present for water at 25° C. is in the order of 1×10⁻⁶ kg/m·sec·Pa. The permeability of membranes according to the present invention is in the range of 0.1×10⁻⁶ to 5×10⁻⁶ kg/m·sec·Pa.

The membranes of the invention are extremely biocompatible in vitro cell cultures with macrophages and fibroblasts owing to the observation of cell adhesion, cell growth, cell vitality and cell activation, and of the production of extracellular proteins and cytokines.

The mechanical properties and the degradability may be changed almost independently from each other. This combination of membrane properties is unique. Since membranes performing a separation process usually are also mechanically stressed, their mechanical characteristics are also of importance. A typical membrane (as e.g. FIG. 6) has a mechanical performance in the range of the values indicated in the following table:

TABLE 1

| Mechanical | |
|---|---|
| properties | Mean ± SD |
| Tensile strength | 0.15 ± 0.04 |
| (km) | |
| Elongation to break | 115.59 ± 46.31 |
| (%) | |
| Modulus of | 0.42 ± 0.05 |
| elasticity (km) | |

Membranes according to the present invention comprise a biocompatible block copolymer. Suitable biocompatible block copolymers have been described in EP 0 696 605 and EP 1 498 147 which are both incorporated herein by reference.

This block copolymer has at least two block units obtainable by linear polycondensation in the presence of diisocyanate, diacid halide or phosgene of a first block unit selected from the group consisting of a diol (I) and an α,ω-dihydroxy-polyester (IV) with a second block unit selected from the group consisting of the same diol (I), a further α,ω-dihydroxy-polyester (II), a α,ω-dihydroxy-polyether (III) and the same α,ω-dihydroxy-polyester (IV).

The diol (I) is obtainable by transesterification of poly-[(R)-(3)-hydroxybutyric acid] or copolymers thereof with 3-hydroxyvaleric acid with ethylene glycol.

The α,ω-dihydroxy-polyester (II) is obtainable by ring-opening polymerization of cyclic esters selected from the group consisting of (L,L)-dilactide, (D,D)-dilactide, (D,L)-dilactide, diglycolide or mixtures thereof, or lactones selected from the group consisting of β-(R)-butyrolactone, β-(S)-butyrolactone, β-rac-butyrolactone and ε-caprolactone or mixtures thereof.

The α,ω-dihydroxy-polyether (III) is selected from the group consisting of α,ω-dihydroxy-poly(oxytetramethylene), α,ω-dihydroxy-poly(oxyethylene) and copolymers of ethylene glycol and propylene glycol.

The α,ω-dihydroxy-polyester (IV) can be obtained by transesterification of α,ω-dihydroxy[oligo(3-(R)-hydroxybutyrate)ethylene-oligo(3-(R)-hydroxybutyrate)] (I), which is referred to hereinafter as PHB diol (IV), with diglycolide dilactide or caprolactone or mixtures thereof, the trans-esterification preferably being carried out in the presence of a catalyst. In the following reaction scheme, m is 1 to 50, n is 1 to 50, x+y is 1 to 50.

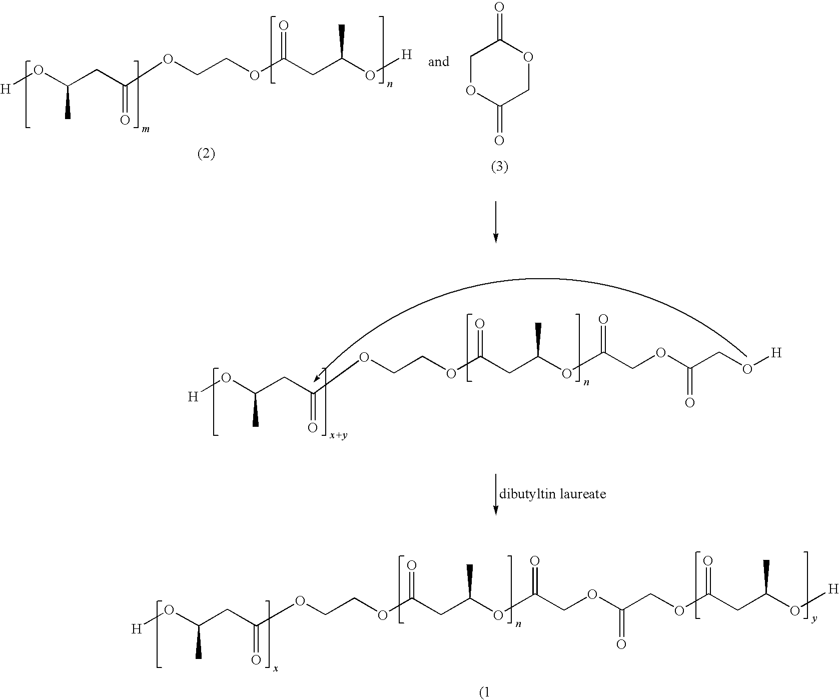

Preferred catalysts are transesterification catalysts in particular based on tin, e.g. dibutyltin dilaurate. The diol preferably has a molecular weight of from 500 to 10'000 daltons. The diol (1) preferably has a total glycolide content of up to 40 mol %, particularly preferably up to 30 mol %. A preferred diol of the invention is α,ω-dihydroxy[oligo(3-R-hydroxybutyrate)-stat-glycolide)ethyleneoligo(3R)-hydroxybutyrate-stat-glycolide) or the corresponding stat-lactide or stat-caprolactate compounds If dilactide or caprolactone is used instead of diglycolide.

Further suitable α,ω-dihydroxypolyesters (II) are oligomers of α-, β-, γ- and co-hydroxy carboxylic acids and their cooligomers which are obtained by ring-opening polymerization of cyclic esters or lactones. Preferred cyclic esters of this type are (L,L)-dilactide, (D,D)-dilactide, (D,L)-dilactide, diglycolide or the preferred lactones such as β-(R)-butyrolactone, β-(S)-butyrolactone, β-rac-butyrolactone and ε-caprolactone or mixtures thereof. The ring opening takes place with aliphatic diols such as ethylene glycol or longer-chain diols. The molecular weight of the resulting macrodiol is determined by the stoichiometrically employed amount of these diols.

The ring-opening polymerization of the cyclic esters or lactones preferably takes place without diluent in the presence of a catalyst, for example SnO(Bu)₂ at 100° C. to 160° C. The resulting macrodiols have molecular weights of about 300-10'000 daltons. The macrodiols prepared from mixtures of cyclic esters or lactones have a microstructure which depends on the amount of catalyst and which is statistical or alternating in the distribution of the monomeric components between block form. The distributions statistics have an influence on the physical properties. Examples of such esters which are obtained by ring-opening polymerization of cyclic esters and lactones in the presence of a catalyst and which can be used to prepare the block copolymers are α,ω-dihydroxy-[poly(L-lactide)-ethylene-poly(L-lactide)]; α,ω-dihydroxy-[oligo(3-(R)-hydroxybutyrate-ran-3-(S)-hydroxybutyrate)-ethylene-oligo(3-(R)-hydroxybutyrate-ran-3-(S)-hydroxybutyrate)]; α,ω-dihydroxy-[oligo(glycolide-ran-ε-caprolactone)-ethylene-oligo(glycolide-ran-ε-caprolactone)]; α,ω-dihydroxy-[oligo(L)-lactide-ran-ε-caprolactone)-ethylene-oligo(L)-lactide-ran-ε-caprolactone)]; α,ω-dihydroxy-[oligo(L)-lactide-ran-glycolide)-ethylene-oligo(L)-lactide-ran-glycolide)]; α,ω-dihydroxy-[oligo(3-(R)-hydroxybutyrate-ran-3-(S)-hydroxybutyrate-ran-glycolide)-ethylene-oligo(3-(R)hydroxybutyrate-ran-3-(S)hydroxybutyrate-ran-glycolide); α,ω-dihydroxy-[oligo-3-(R)-hydroxybutyrate-ran-3-(S)-hydroxybutyrate-ran-L-lactide-ethylene-oligo(3-(R)-hydroxybutyrate-ran-(S)-hydroxybutyrate-ran-L-lactide)] and α,ω-hydroxy-[oligo(3-(R)-hydroxybutyrate-ran-3-(S)-hydroxybutyrate-ran-ε-caprolactone)ethylene-oligo(3-(R)-hydroxybutyrate-ran-3-(S)-hydroxybutyrate-ran-ε-caprolactone)].

The ring-opening polymerization for preparing these macrodiols can also take place without catalyst. Diisocyanates suitable for preparing the polyurethane variant of the block copolymers are in particular hexamethylene diisocyanate, 2,2,4-trimethylhexamethylene diisocyanate, cyclohexyl 1,4-diisocyanate, cyclohexyl 1,2-diisocyanate, isophorone diisocyanate, methylenedicyclohexyl diisocyanate and L-lysine diisocyanate methyl ester.

Diacid halides particularly suitable for preparing the polyester variant of the block copolymers are those of oxalic acid, malonic acid, succinic acid, glutaric acid, adipic acid, pimelic acid, suberic acid, azelaic acid, trimethyladipic acid, sebacic acid, dodecanediacid, tetradecanedioic acid and hexadecanedioic acid.

Reaction to give the polymer of the invention takes place almost quantitatively. It has moreover been found that incorporation of the dilactide, diglycolide and/or caprolactone units results in the polymers of the invention being soluble in methylene chloride. It is thus possible to remove impurities by filtration. A cost-effective process with which the polymer of the invention can be prepared with high purity is provided thereby.

In preferred embodiments the first block unit is the α,ω-dihydroxy-polyester (IV) and the second block unit is either the diol (I), the α,ω-dihydroxy-polyester (II), α,ω-dihydroxy-polyether (III) or the same α,ω-dihydroxy-polyester (IV).

A particularly preferred block copolymer is poly[poly[α,ω-dihydroxy-[oligo(3-(R)-hydroxybutyrate)-stat-glycolide)-ethylene-oligo-(3-(R)-hydroxybutyrate-stat-glycolide)]alt-2,2,4-trimethylhexamethylene 1,6-diisocyanate]-co-poly[dihydroxy[oligo-glycolide-ran-ε-caprolactone)-ethylene-(oligo-glycolide-ran-ε-caprolactone)]alt-2,2,4-trimethylhexamethylene 1,6-diisocyanate] of the formula

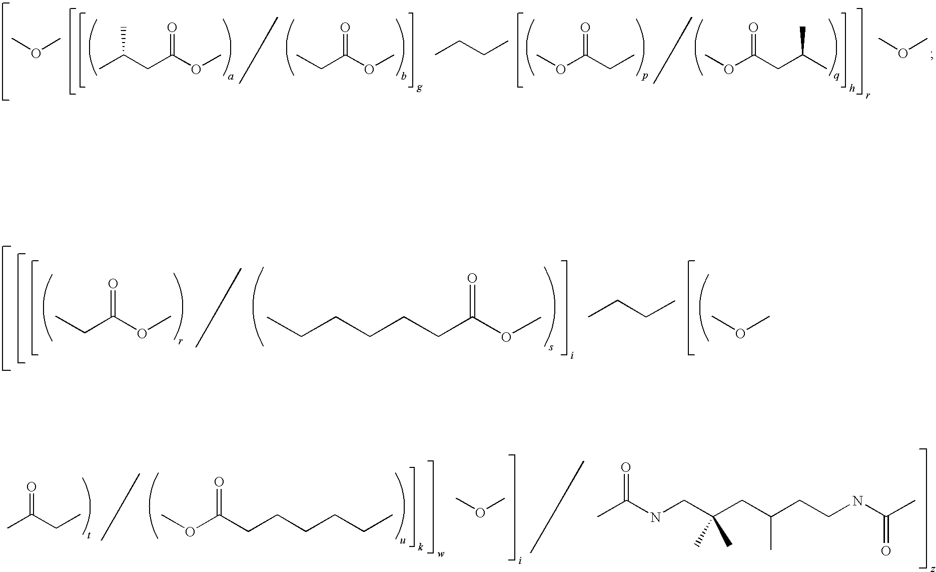

where a=1 to 50, b=1 to 10, g=1 to 50, h=1 50, i=1 to 50, k=1 to 50, w=1 to 50, p=1 to 10, q=1 to 50, r=1 to 10, s=1 to 50, t=1 to 10, u=1 to 50 and z=1 to 50. Further preferred polymers are identical to the abovementioned with the exception that the glycolide unit of the polymer is replaced by the corresponding lactide or caprolactone.

The membranes comprising glycolide units which are particularly preferred are those degradable in five to six days within the human or animal body. Further preferred membranes are those whose degradation takes place over months or years. The rate of degradation depends primarily on the number of diglycolide or glycolide units. On storage in a neutral buffer solution at 37° C., the molecular weight decreases with time as a function of the glycolide content. The use of dilactide or caprolactone units does not change the rate of degradability of the polymers of the invention in the body.

The membranes can comprise more than one layer. In a preferred embodiment the membrane comprises only single layer.

It has been found that the membrane according to the present invention has an exceptionally good biocompatibility. In addition, it is possible through the incorporation of the glycolide or diglycolide units to control the hydrolytic and biological rate of degradability of the membrane. The degradability of the block copolymer outside the body can be increased, besides the incorporation of glycolide or diglycolide units, by (L,L)-dilactide, (D,D)-dilactide, (D,L)-dilactide or mixtures thereof.

In a preferred embodiment the membranes have a service time. This service time may be defined as the time which elapses from the time point in which the membrane comes to the first time into contact with water and the point in time, when the mechanical properties of the material begins to drop down, and the properties of the membrane become insufficient to fulfill its task. This is, when the membrane begins to loose mass, is getting brittle or the pore size and shape alters. These changes go in parallel with the changes of the parameter in table 1 above. The service time of the said membranes may be 5 days up to 2 years. Preferably, the service life time of a membrane according to the present invention is between 14 to 28 days. This range is suitable for tissue engineering. On a molecular basis this is the time point, when the macromolecules, building up our material drop below a average molecular weight of about 10 000 Da.

The present invention also relates to the use of membranes of the invention as surgical aids.

Membranes of the present invention can be employed as surgical aids or can be comprised in surgical aids. A major advantage of the use of membranes as surgical aids or implants is their controllable degradability. Because this degradability can be tailored according to the needs of the particular situation a second surgery, in order to remove an implant that is no longer needed, can be avoided. Such second surgeries are a prominent source of complications, e.g. causing unwanted inflammation, infections etc., besides the fact that they drive up costs.

Since the membranes can be prepared in various forms, e.g. flat, tubular or as capsules, the membranes provide an excellent flexibility in terms of possible uses. For instance, the implants may be in the form of a tube. The tube may be rigid or flexible. The tubes may have circular, elliptical and polygonal cross sections.

The implant material may have a porous structure for particular uses. It is possible with the implants of the invention to regenerate a functional vessel wall or a nerve. It is possible by a coating with functional vessel cells (endothelial cells) to avoid a thrombotic occlusion on long-term use, i.e. the biocompatible polymer can be a replacement. The implant may also have a capsule shape to receive pharmaceutical active substances or diagnostics also in the form of particles.

In a preferred embodiment the membrane of the present invention comprises at least one pharmaceutically active compound or diagnostic aid. Such compounds include hormones, enzymes, cytokines, growth factors, anti-inflammatory drugs, e.g. steroids or non steroidal anti-inflammatory drugs (NSAIDs) and the like. These compounds can be entrapped within the membrane or they can be covalently bound to the membrane. Preferably, they are covalently bound to the membrane.

In addition, further possible uses in appropriate physical and or biological form are in medical dental, micro- or nanotechnologies.

The present invention also relates to a method for preparing a membrane.

A method for preparing a membrane according to the present invention comprises the following steps, first a biocompatible block copolymer is dissolved in a suitable solvent, e.g. dioxane, second the block copolymer solution is applied on a carrier. If the membrane to be prepared is flat, it a glass plate can be used as a carrier. Third, after the block copolymer solution has been applied on the carrier, said carrier is immersed in a non-solvent which is miscible with the solvent, resulting in the porous membrane, the pores of which having size in the range of 0.2 to 20.0 μm.

The carrier which is employed depends on the desired shape of the membrane. Apart from glass plates for flat membranes, tubular forms can be used to prepare membranes of cylindrical shape.

Instead of immersing the carrier with the applied block copolymer solution in the non-solvent, the latter can also be sprayed on said carrier.

Suitable solvents for the block copolymers are dioxane, chloroform, dimethylcarbonate and butanon. A preferred solvent is 1,4-dioxane. It can easily removed from the polymer and has the least toxic side effects.

As non-solvents may be used water, methanol and ethanol.

A preferred non-solvent is water. A preferred solvent/non-solvent pair is 1,4-dioxane and water.

The present invention also relates to sheet-like structures comprising a membrane.

Said sheet-like structure may comprise a membrane and for instance a mesh. The fibers forming the mesh further enhance the mechanical stability of the structure.

### Example 1

### Manufacturing a Membrane

To obtain a membrane, the molecular weight (Mw) of the biocompatible block-copolymer must exceed a lower limit 50'000 Da<Mw. Otherwise the result will be a transparent film and not a membrane. The polymer is dissolved in dioxane at a concentration of 6-25% wt/wt. Four coagulation baths are used successively. The first one with EtOH (or an alternative non-solvent), the second with MeOH (or an alternative non-solvent), the third one with distilled water (in which few detergent is added) and the last one with distilled water. On a clean glass plate, with help of a doctor's blade allowing to doctor a film of 500 μm is used. It is pushed gently over the glass' surface. The glass plate with the layer of polymer solution on it is then contacted with the non solvent in the first bath, where the transparent film becomes opaque resulting in a porous membrane. The film is then successively washed in the remaining bath's and at the end of the fourth bath, the membrane is dried on air or in the vacuum oven. The resulting pores have a size in the range of 0.2 μm to 20 μm. Alternatively the film which is doctored on the glass plate may first be contacted with non solvent by spraying the non solvent as a fine haze over the surface. This process can be transferred to a enlarged process of membrane production on the surface of a rotating cylinder.

### Production of a Membrane Tube

To receive a perfect membrane tube, the molecular weight of the applied polymer should exceed 70'000 dalton. The polymer is solved in dioxane at a concentration of 25%.

Three baths are necessary: the first one with MeOH cooled down to −20° C. (+/−2° C.). The temperature should be as constant as possible. The second one with EtOH held at 0° C. and the last one filled with distilled water at room temperature.

There are two methods to produce tubes:

The first one is the so called negative method.

A glass tube with the right internal diameter is provided as well as an internal tube (PTFE or glass is needed to prevent the resulting membrane from sticking together)

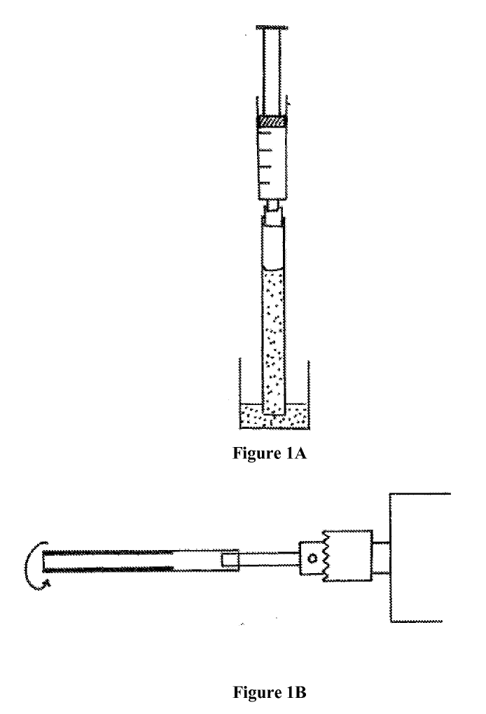

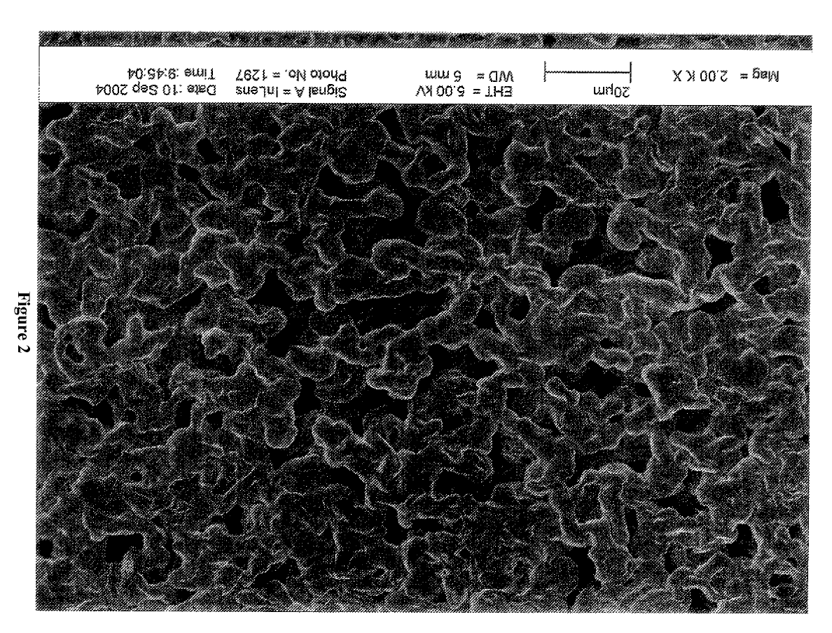

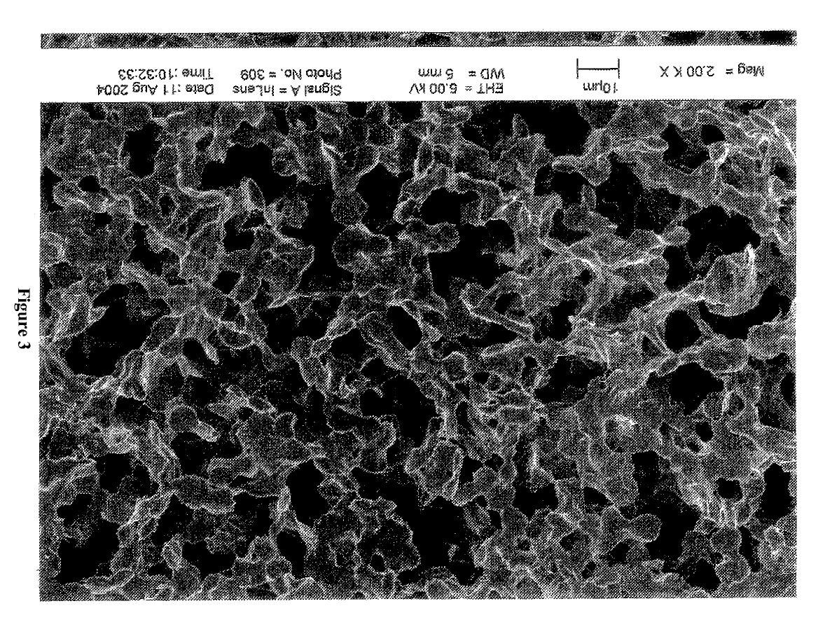

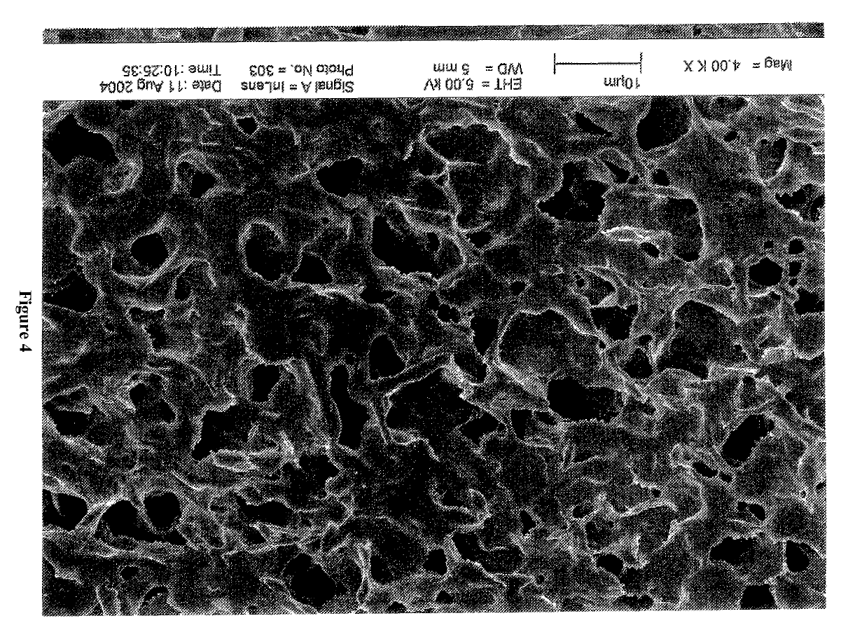

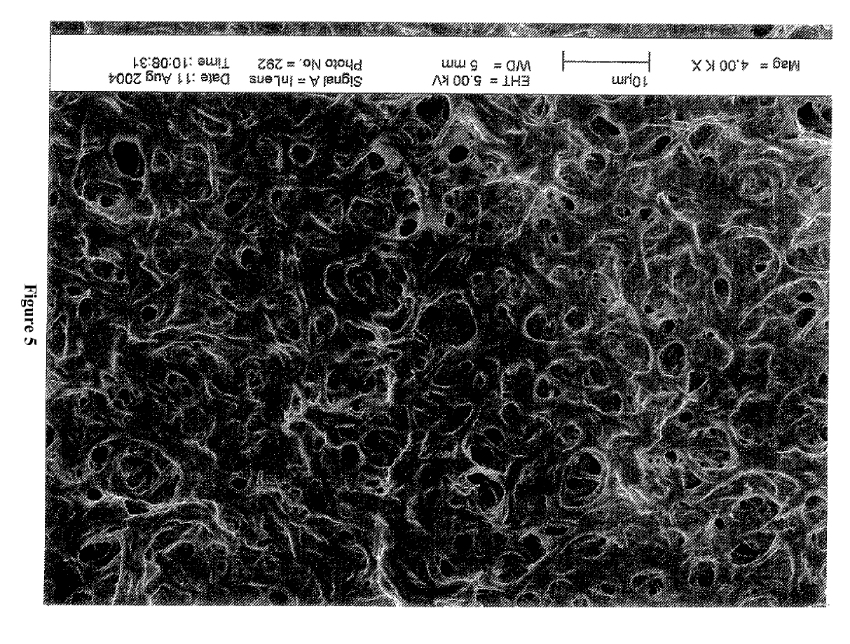

The beforehand prepared solution is sucked into the tube (FIG. 1A). The spare solvent drips out back into the container. The resuming solution adheres on the tube's wall.

The glass tube is now mounted horizontally into a stirring motor and turned at 2000 turns per minute for several seconds (FIG. 1B).

Consequently, the resulting layer on the glass tube's wall is of a homogeneous thickness.

Afterwards, the tube is dropped instantly into the cooled first bath and remains there for approximately five minutes. After that, the tube is transferred for another five minutes into the second bath and finally into the last one (before putting it into the last one, the internal tube has to be adjusted).

The second method is called the positive method.

Here, we directly use a bar with the right diameter:

The prepared polymer solution should be as viscous as possible since it has to adhere on the pole's surface. The bar is dipped into the polymer solution and immediately put into the first bath where the thin layer of the dioxane solution freezes instantly. After 30 minutes, it is then transferred into the second bath and finally put into the third where the membrane either tears or dismantles from the bar.

### Porosity and Surface Structure

The porosity can be adjusted by altering the solution's concentration. Starting with a solution of 10% and ending with one with over 25% polymer solved in dioxane.

It is shown that an increasing concentration comes along with a reduction of the pore size.

### Combination of Membrane and Electrospun Fibres

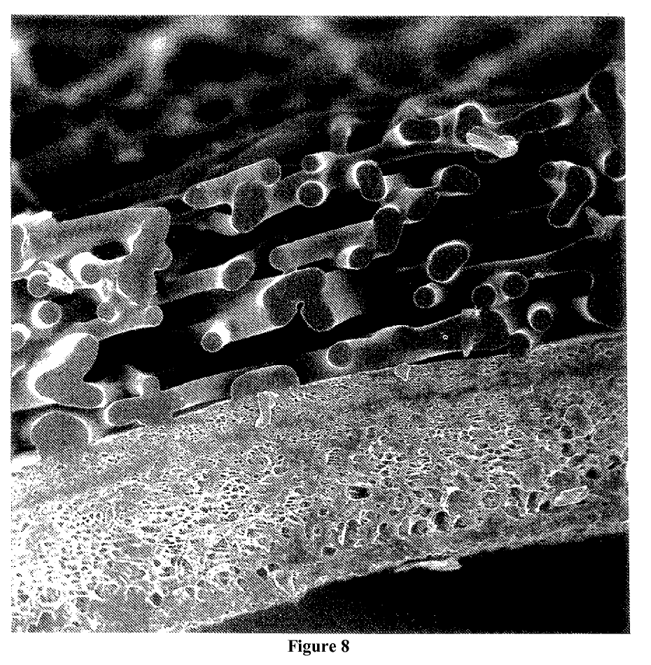

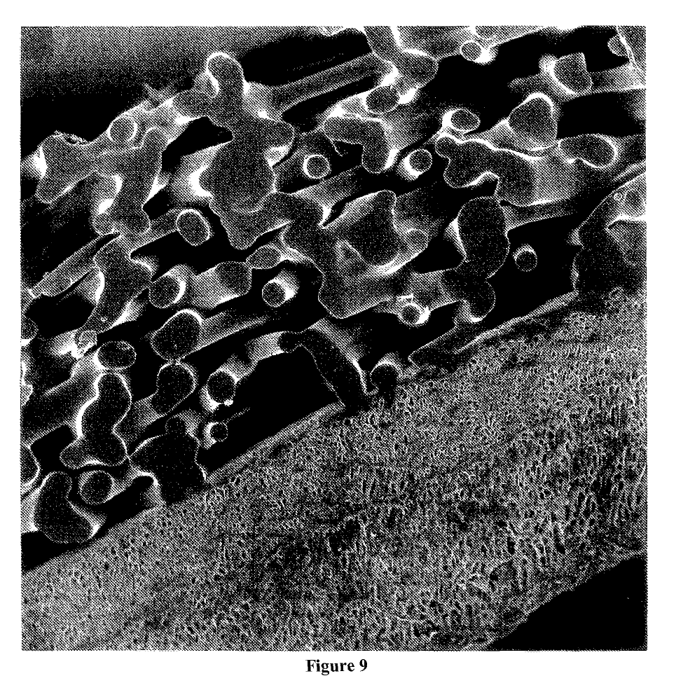

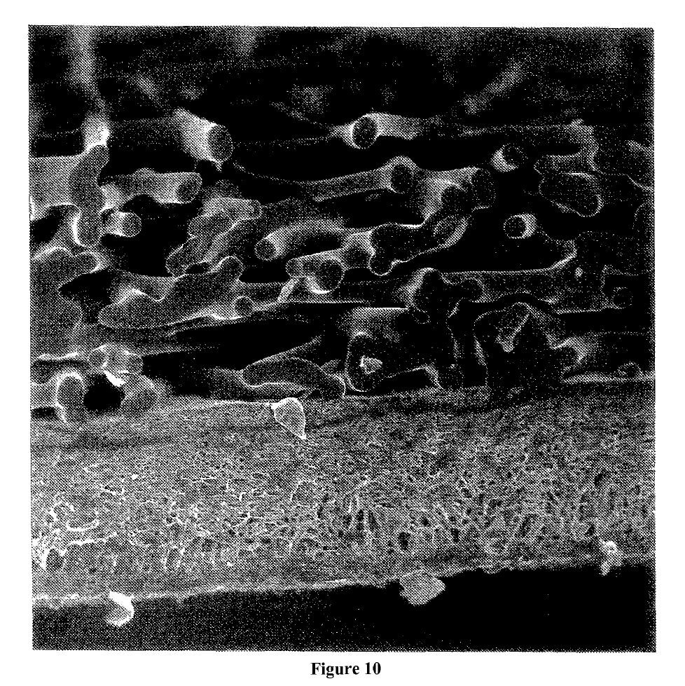

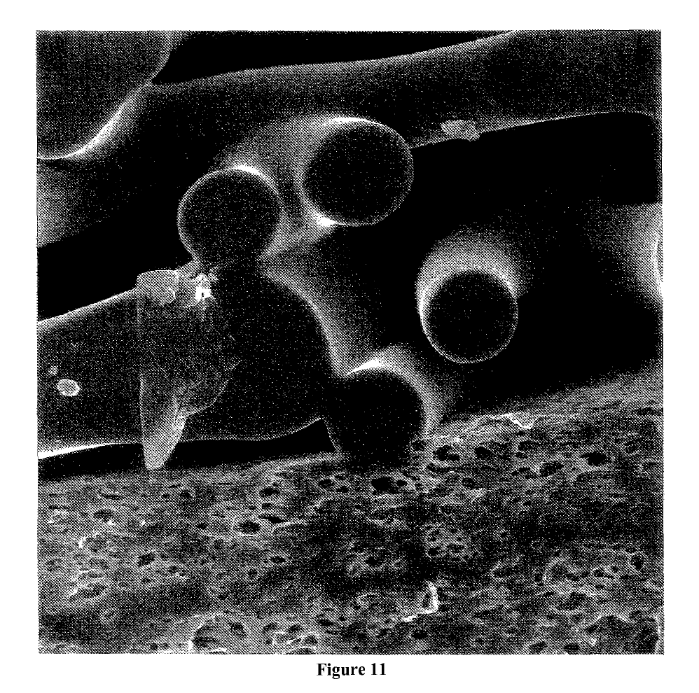

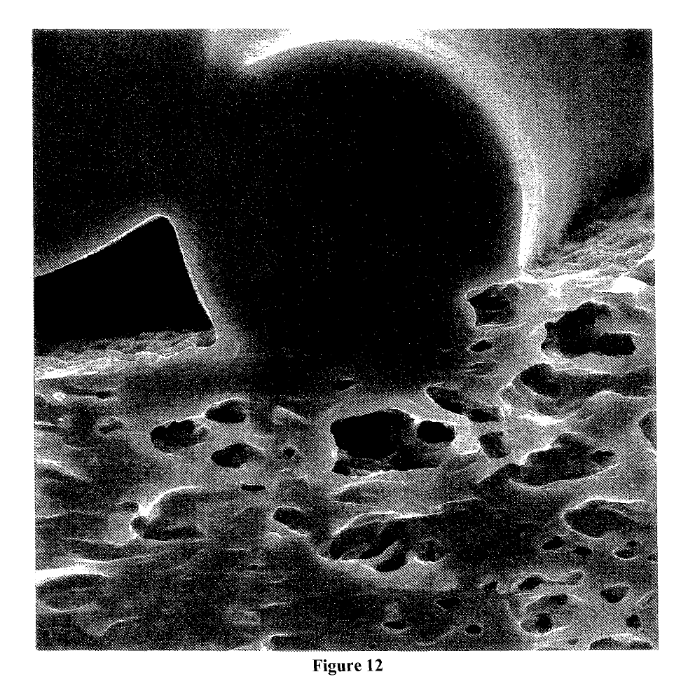

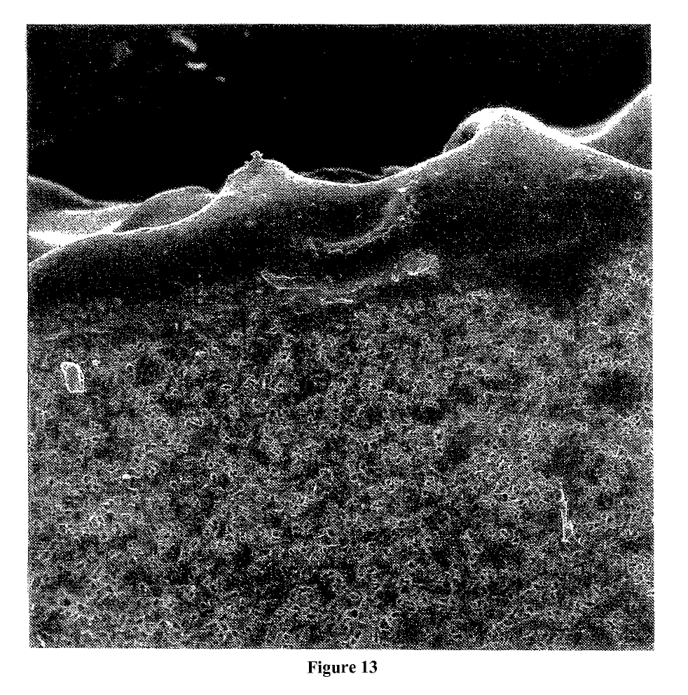

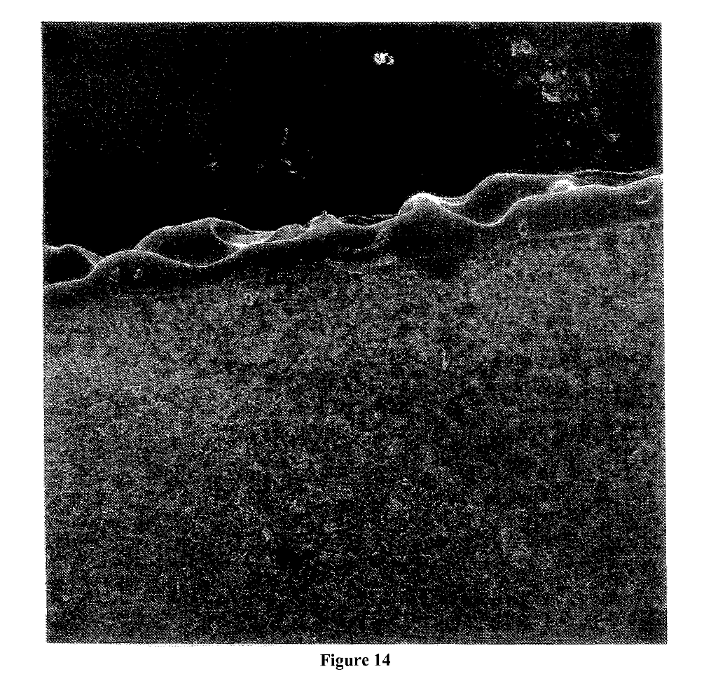

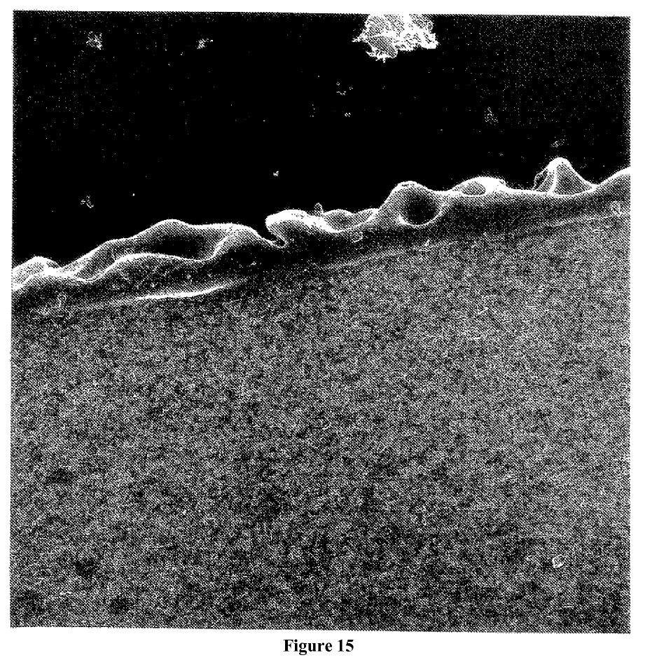

The resulting membranes are homogeneously porous but not very strong. Therefore, a combination of membrane and a strong electrospun fibre network was aspired. (With the same polymer). Typical examples of such sheet-like structures comprising a membrane and a mesh of fibres are shown in FIGS. 8 to 12.

### Membrane Bags

Several applications demand for a closed structure akin to a bag wherein cells or other material can be stored.

Bags can be obtained by fusing two congruent membranes together. A heated bar (approx 150° C.) was used to melt the edges together. In order to prevent the membrane from melting, it is moistened with distilled water.

### Example 2

Mass transfer coefficients D were determined for a membrane at room temperature and water, with a series of fluorescent polysaccharides (FITC-Ficoll) with different molecular weights The following values were found for a 65 μm thick membrane:

| | | | Mass Transfer |
|---|---|---|---|
| | | | coefficient D |
| Tracer | MW | N | in mm²/sec |
| Iohexol | 821 | 6 | 8.3 ± 1.6 × 10 − 4 |
| FITC-Ficoll 16 | 5000 | 2 | 5.3 ± 1.6 × 10 − 4 |
| FITC-Ficoll 30 | 22000 | 2 | 3.2 ± 0.5 × 10 − 4 |
| FITC-Ficoll 50 | 72000 | 2 | 2.4 ± 0.1 × 10 − 4 |
| FITC-Ficoll 120 | 561000 | 2 | 2.0 ± 0.5 × 10 − 4 |

### Example 3

### Fluorescence Microscope Analysis of Cell Seeded Polymers

A live-death stain was performed on the polymer variants.

Fluorescein diacetate (FDA) and ethidium bromide (EB) were used to indicate live and dead cells respectively. At time points of 2 d, 4 d and 12 d, two wells for each variant and control were examined. The medium was removed. Cell seeding of polymer variants and the cells were washed twice with 200 μl sterile phosphate buffered saline (PBS). 2 ml 70% ethanol was added to the control cells and incubated for 5 minutes to act as a negative control.

After this time the ethanol was removed. The cells were then stained with Fluorescein-diacetate (FDA) (2.5 μl/ml) and ethidium bromide (EB) (10 μl/ml) in PBS for 1 minute at room temperature. Staining was removed and washed twice with sterile PBS. Live and dead cells were analysed using fluorescence microscopy.

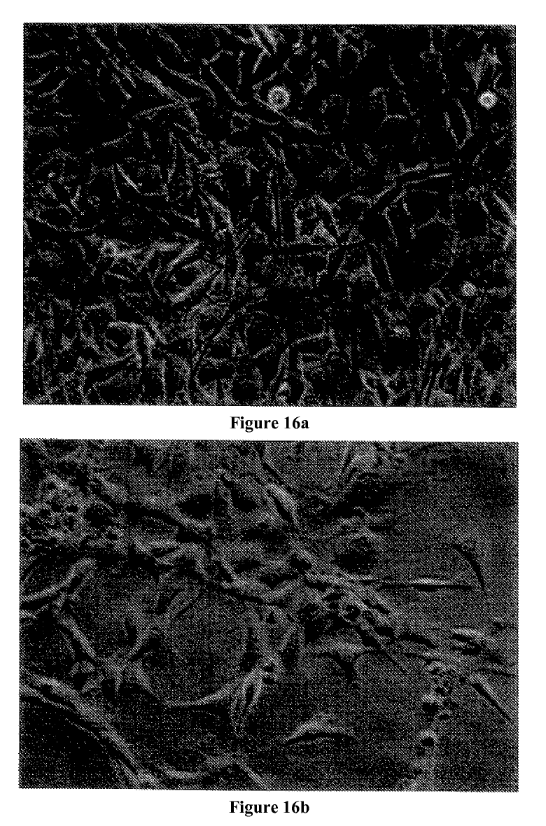

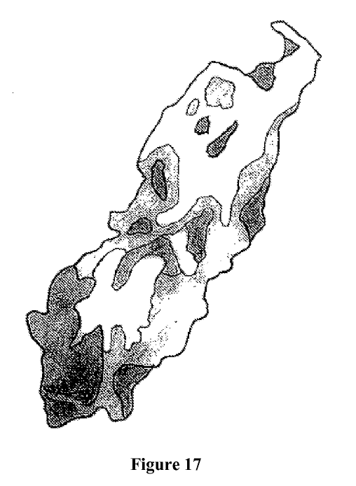

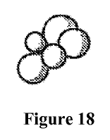

Cytotoxicity test: Images of the cells after 72 h are detailed in FIG. 16. All variants exhibit good response to the eluate compared to the control indicating that any released by-products after 24 h are not very toxic to the cells. Continued good cell growth was observed at further time points (not shown).

1. Membrane comprising a biocompatible block copolymer having at least two block units obtainable by linear polycondensation in the presence of diisocyanate, diacid halide or phosgene of first block unit selected from the group consisting of a diol (I) and an α,ω-dihydroxy-polyester (IV) with a second block unit selected from the group consisting of the same diol (I), an α,ω-dihydroxy-polyester (II), an α,ω-dihydroxy-polyether (III), and the same α,ω-dihydroxy-polyester (IV),
wherein the diol (I) is obtainable by transesterification of poly-[(R)-(3)-hydroxybutyric acid] or copolymers thereof with 3-hydroxyvaleric acid and ethylene glycol,
wherein the α,ω-dihydroxy-polyester (II) is obtainable by ring-opening polymerization of cyclic esters selected from the group consisting of (L,L)-dilactide, (D,D)-dilactide, (D,L)-dilactide, diglycolide or mixtures thereof, or lactones selected from the group consisting of β-(R)-butyrolactone, β-(S)-butyrolactone, β-rac-butyrolactone and ε-caprolactone or mixtures thereof,
wherein the α,ω-dihydroxy-polyether (III) is selected from the group consisting of α,ω-dihydroxy-poly(oxytetramethylene), α,ω-dihydroxy-poly(oxyethylene) and copolymers of ethylene glycol and propylene glycol,
wherein the α,ω-dihydroxy-polyester (IV) is obtainable by trans-esterification of the diol (I) with diglycolide and/or dilactide and/or caprolactone or mixtures thereof, and
characterized in that the membrane comprises regularly distributed pores.

2. Membrane according to claim 1, wherein the first block unit is the α,ω-dihydroxy-polyester (IV) and the second block unit is the diol (I).

3. Membrane according to claim 1, wherein the first block unit is the α,ω-dihydroxy-polyester (IV) and the second block unit is the α,ω-dihydroxy-polyester (II).

4. Membrane according to claim 1, wherein the first block unit is the α,ω-dihydroxy-polyester (IV) and the second block unit is the same α,ω-dihydroxy-polyether (IIII).

5. Membrane according to claim 1, wherein the first block unit is the α,ω-dihydroxy-polyester (IV) and the second block unit is the α,ω-dihydroxy-polyester (IV).

6. Membrane according to claim 1, wherein the pores have a size in the range of 0.2 to 20.0 μm.

7. Membrane according to claim 1, wherein said membrane is permeable or semipermeable.

8. Membrane according to claim 1, wherein the permeability is in the range of 0.1×10⁻⁶ to 5×10⁻⁶ kg/m·sec·Pa.

9. Membrane according to claim 1, wherein the block copolymer is poly[poly[α-ω-dihydroxy-[oligo(3-(R)-hydroxybutyrate)-stat-glycolide)-ethylene-oligo-(3-(R)-hydroxybutyrate-stat-glycolide)]alt-2,2,4-trimethylhexamethylene-1,6-diisocyanate]-co-poly-[dihydroxy[oligo-glycolide-ran-ε-caprolactone)-ethylene-(oligo-glycolide-ran-ε-caprolactone)]alt-2,2,4-trimethylhexamethylene-1,6-diisocyanate].

10. Membrane according to claim 1, wherein the block copolymer is poly[poly[α-ω-Dihydroxy-[oligo(3-(R)-hydroxybutyrat)-co-ε-caprolacton)-ethylen-oligo-(3-(R)-hydroxybutyrat-co-ε-caprolacton)]alt-2,2,4-trimethylhexamethylene-1,6-diisocyanat]-copoly[dihydroxy[oligo-glykolid-ran-ε-caprolacton)-ethylen-(oligo-glykolid-ran-ε-caprolacton)]alt-2,2,4-trimethylhexamethylene-1,6-diisocyanat].

11. Membrane according to claim 1, wherein said membrane comprises a single layer.

12. Membrane according to claim 1, wherein said membrane is hydrolytically degradable and has a controllable service life time and keeps its performance from 5 days to 2 years.

13. Membrane according to claim 1, wherein said membrane has a tubular form.

14. Membrane according to claim 1, wherein said membrane comprises at least one pharmaceutically active compound or diagnostic aid.

15. A surgical aid intended to be fixed in and on the human or animal body, comprising said membrane as claimed in claims 1.

16. Method for preparing a membrane as claimed in claim 1, wherein
a) a biocompatible block copolymer is dissolved in a solvent; and
b) said block copolymer solution is applied on a suitable, shaped carrier; and
c) said carrier is immersed in a non-solvent which is miscible with the solvent, resulting in the membrane comprising pores having a size in the range of 0.2-20.0 μm.

17. Method according to claim 16, wherein the non-solvent is sprayed on said carrier.

18. Method according to claim 16, wherein the solvent is selected from the group consisting of dioxane, chloroform, dimethylcarbonate and butanon.

19. Method according to claim 16, wherein the solvent is 1,4-dioxane.

20. Method according to claim 16, wherein the non-solvent is selected from the group consisting of water, methanol and ethanol.

21. Method according to claim 16, wherein the non-solvent is water.

22. Method for preparing a membrane as claimed in claim 1, wherein
a) a biocompatible block copolymer is dissolved in a solvent; and
b) said block copolymer solution is applied on a suitable, shaped carrier; and
c) said carrier is immersed in a non-solvent which is miscible with the solvent, resulting in the membrane comprising uniformly distributed pores.

23. A medical implant or a surgical aid comprising a membrane according to claim 1.

24. Sheet-like structure, wherein said structure comprises a membrane according to claim 1.

25. Sheet-like structure, comprising a membrane according to claim 1 and a mesh.

26. Membrane according to claim 6, wherein the pores have a size in the range of 0.2 to 10.0 μm.

27. Membrane according to claim 12, wherein said membrane keeps its performance from between 14 and 28 days.
